(19) United States
(12) Patent Application Publication
(10) Pub. No.: US 2024/0216422 A1
(43) Pub. Date: Jul. 4, 2024

(54) REMEDIATION OF ALL DISEASES CAUSED BY RADIATION RELATED REACTIVE ION SPECIES ARE ALL CORRECTABLE BY H2S - SO2 GENERATED BY FDA-STS SODIUM THIOSULFATE, NA2S203

(71) Applicant: Frederick J. Sawaya, southfield, MI (US)

(72) Inventor: Frederick J. Sawaya, southfield, MI (US)

(21) Appl. No.: 18/482,872

(22) Filed: Oct. 7, 2023

Related U.S. Application Data
(60) Provisional application No. 63/436,671, filed on Jan. 2, 2023.
(60) Provisional application No. 63/501,905, filed on May 12, 2023.

(51) Int. Cl. A61K 33/04; A61K 31/375; A61K 31/616

(52) U.S. Cl. CPC A61K 33/04; A61K 31/375; A61K 31/616

(57) ABSTRACT

A composition and method for addressing the presence of reactive oxygen species (ROS), reactive nitrogen species (RNS), and/or reactive sulfur species (RSS) abnormalities for the treatment, inhibition, and/or attenuation of disease states in a patient in need thereof. The method includes administering a sulfur-containing compound, such as sodium thiosulfate. A catalytic component can also be added to the composition to augment the rate of sodium thiosulfate action within the body.

### CROSS-REFERENCE TO RELATED APPLICATIONS

This application claims priority from a U.S. Provisional Patent Appl. Ser. No. 63/436,671 filed on Jan. 2, 2023, and a U.S. Provisional Patent Appl. Ser. No. 63/501,905 filed May 12, 2023, both of which are hereby incorporated by reference herein in its entirety.

### FIELD OF INVENTION

The present invention relates to a composition and method for treatment, inhibition, or attenuation of a disease in a patient, and more particularly, the present invention relates to a composition and method for the treatment, inhibition, or attenuation of diseases caused by nuclear radioactive events or medical treatments causing abnormalities in reactive oxygen species, reactive nitrogen species, and reactive sulfur species.

### BACKGROUND

Reactive oxygen species (ROS), reactive sulfur species (RSS), and reactive nitrogen species (RNS) play an important role in biological responses in humans. ROS, RSS, and RNS are signaling molecules found in the body and are produced by external and internal bodily stresses. Some human disease states are characterized by the presence of abnormal links ROS, RSS, and/or RNS, or the presence of excess or defiencies of normal ROS, RSS and/or RNS, or the presence of ROS, RSS, and/or RNS incorrect locations in human cells. These three ions together balance every chemical species in the cells in the body, but H2S is the final word on the location number location and kind of chemicals allowed in humans animals, agriculture plants, and waterways.

A need is therefore appreciated for a composition and method for addressing the presence of these ROS, RSS, and/or RNS abnormalities.

### SUMMARY OF THE INVENTION

The following presents a simplified summary of one or more embodiments of the present invention to provide a basic understanding of such embodiments. This summary is not an extensive overview of all contemplated embodiments and is intended to neither identify key or critical elements of all embodiments nor delineate the scope of any or all embodiments.

The principal object of the present invention is therefore directed to a composition and method for addressing the presence of ROS/RSS/RNS abnormalities for the treatment, inhibition and/or attenuation of disease states.

### BRIEF DESCRIPTION OF DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 schematically shows cleaving of a disulfide bond with a thiol group.

FIG. 2 schematically shows a cell subject to ionizing radiation.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing embodiments only and is not intended to be limiting to embodiments of the invention. As used herein, the singular forms “a”, “an” and “the” are intended to include the plural forms as well, unless the context clearly indicates otherwise. It will be further understood that the terms “comprise”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention since the scope of the invention will be best defined by the allowed claims of any resulting patent.

In the following detailed description Sodium Thiosulfate, STS, and Na₂S₂O₃ are used interchangeably referring to sodium thiosulfate. The term S—S bond refers to disulfide bonds, H₂S refers to Hydrogen sulfide and SO2 refers to sulfur dioxide. Here, H2SO3 refers to sulfurous acid.

Disclosed is a composition and method for the treatment, inhibition, and/or attenuation of various disease states. The disclosed composition and method can be useful in a number of diseases the occurrence and development of which may involve the imbalances in ROS, RNS, and/or RSS in the body. The imbalances can be in the production, accumulation, or distribution of the ROS, RNS, and/or RSS. Also, disclosed is a method of restoring the balances of the ROS, RNS, and/or RSS in the body. Also, disclosed is a mechanism of action of the disclosed composition in addressing the ROS, RNS, and/or RSS imbalances in the body. Various factors may contribute to the imbalances in the ROS, RNS, and/or RSS in the body, for example, external stress, oxidative stress, ionizing radiation stress, and the like.

The composition provides a sulfur containing compound which is used in solid and liquid forms. The sulfur containing compound is sodium salt of sulfur. The sulfur containing compound is sodium thiosulfate (STS). The composition comprises sodium thiosulfate along with necessary additives.

In one implementation, disclosed is a composition comprising a sulfur-containing compound, and in particular sodium thiosulfate (Na₂S₂O₃, or “STS”). STS is a colorless crystalline compound that commonly exists as the pentahydrate, Na₂S₂O₃·5H₂O, an efflorescent, monoclinic crystalline substance also called sodium hyposulfite. Sodium thiosulfate is produced chiefly from liquid waste products of sodium sulfide or sulfur dye manufacture. In the laboratory, this salt can be prepared by heating an aqueous solution of sodium sulfate with sulfur or by boiling aqueous sodium hydroxide and sulfuric acid according to the following Equation 1:

$$6 NaOH + 4 S \to 2 {Na}_{2} S + {Na}_{2} {S}_{2} {O}_{3} + 3 {H}_{2} O \qquad ( 1 )$$

It has been found out that STS is safe for humans and is effective in the treatment of cyanide overdose, eclampsia, and kidney stones, wherein it acts through a different mechanism of action. The applicant found methods through which the STS can help restore the balances of ROS, RNS, and/or RSS in the body.

Also disclosed are different formulations for STS for administering the STS in the body through different routes. Examples of such formulations include tablets, powders, capsules, solutions, creams, pastes, or lotions. STS in suitable formulation can be delivered through a variety of routes including intravenously, orally, rectally, sublingually, vaginally, topically, or inhalation, ophthalmic, intravesical, or applied to waterways, ice.

Depending on the conditions, STS breaks down in the human body to form H₂S, SO₂, and/or S (sulfur) as shown in Equation 2 below. The breakdown is reversible, meaning STS can reform under certain conditions.

$${Na}_{2} {S}_{2} {O}_{3} \to {H}_{2} S ( g ) + {SO}_{2} ( g ) + S ( s ) \qquad ( 2 )$$

For instance, humans produce the enzyme thiosulfate reductase in the liver, kidneys, and testes. It is also believed to be produced within the mitochondria of the human cell itself. When STS encounters this enzyme, the reduction product is H₂S according to the reaction shown below in Equation 3:

$${S}_{2} {O}_{3}^{- 2} + 2 {H}^{+} + 2 {e}^{-} \to {HS}^{-} + {HSO}_{3}^{-} \qquad ( 3 )$$

Both H₂S and SO₂ are naturally present in the human body. In general, the human body regulates the amount of H₂S and SO₂ such that when one is present in large amounts, the other is present in relatively smaller amounts. SO₂ also reacts with water to form H₂S as shown in Equation 4 below, which reaction is upregulated when amounts of H₂S are low.

$$2 {SO}_{2} + 2 {H}_{2} O \to {H}_{2} S + {O}_{2} + {H}_{2} {SO}_{4} \qquad ( 4 )$$

For instance, during mitochondrial respiration the breakdown of acids releases H₂S. Moreover, polysulfides and persulfides can encounter reducing agents, which reduction reaction results in the release of H₂S. However, under certain conditions, the body produces an insufficient amount of H₂S and/or SO₂. For example, if the body is lacking in the amino acid cysteine, the acid breakdown process is inhibited. At the same time, H₂S is known to play an important role in the many critical processes mediated by cysteine, including signaling by ROS, RNS, and/or RSS (discussed below) which can become inhibited if there is not enough H₂S available. Therefore, providing exogenous H₂S and/or SO₂ from STS is advantageous for the reasons that will be discussed below.

H₂S and SO₂ as reaction products have a combination of two unique features: first, they can cleave sulfur-sulfur bonds (S—S) and second, they can enter human cells and human mitochondria. This gives H₂S and SO₂ the ability to destroy amino acids (and thus abnormal DNA/RNA) and proteins, impair the synthesis of abnormal proteins, and impair abnormal DNA replication. H₂S is the most aggressive form of binding sulfur known.

Moreover, H₂S has the unique ability to reversibly combine itself or other sulfurs and polysulfides to create composites, as demonstrated in Equation 5 shown in FIG. 3. This promotes the amount of H₂S available in cells to provide the advantageous effects described herein.

$${H}_{2} S {⟷}^{∓ {e}^{-}} {HS}^{} {⟷}^{∓ {e}^{-}} {H}_{2} {S}_{2} {⟷}^{∓ {e}^{-}} {S}_{2}^{} {⟷}^{∓ {e}^{-}} {S}_{2 } \qquad ( 5 )$$

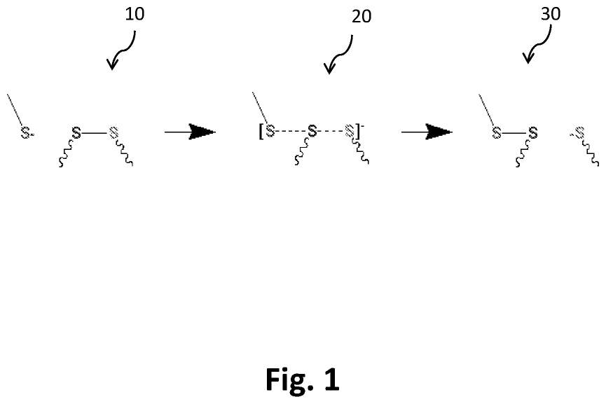

H₂S and SO₂ cleave S—S bonds as follows and as shown in FIG. 1. A thiolate (S—) group attacks one sulfur atom of an S—S bond in step 10, forming a linear intermediate in which the three sulfur atoms interact (shown in step 20). This interaction occurs because the sulfur atom from the thiolate group completes the two missing valence electrons for the sulfur atom which are not present when a sulfur atom is bonded to another sulfur atom. Then, as shown in step 30, the sulfur atom from the thiolate group displaces the second sulfur atom from the S—S bond, cleaving the bond.

H₂S and SO₂ can also bind metal ions including heavy metals and radioactive metals, neutralizing their effects on the body, and allowing them to be cleared.

H₂S can also manage the amounts of ozone present in the body. Too much ozone can have deleterious effects. H₂S reacts with ozone to convert it back to normal oxygen (O₂).

Reactive oxygen species (ROS), reactive sulfur species (RSS) and/or reactive nitrogen species (RNS) are signaling molecules found in the body. ROS are reactive compounds formed from diatomic oxygen (O₂). Examples of ROS include peroxides, superoxide, hydroxyl radicals, singlet oxygen, and alpha oxygen. RSS is a family of sulfur-based chemical compounds that can oxidize and inhibit thiol-proteins and enzymes. They are often formed by the oxidation of thiols and disulfides into higher oxidation states. Examples of RSS include persulfides, polysulfides, thiosulfate, and glutathione. RNS is present in the cells in 1, 2, or 3 forms of Nitrogen commonly used by the body and is considered a signal molecule as well as the S's and O's.

In general ROS, RSS, and RNS signal by reacting with protein cysteines. H₂S and SO₂ participate in the regulation of these signaling processes under normal circumstances. However, ROS, RNS, and/or RSS abnormalities characterizes certain disease states, such as the presence of abnormal ROS, RNS, and/or RSS, the presence of excess amounts of normal ROS, RNS, and/or RSS, and the presence of normal ROS, RNS, and/or RSS in abnormal locations within cells or the body. These abnormalities can in turn interfere with the body's normal signaling processes and can result in the formation of mutagenic proteins and/or abnormal DNA/RNA. One example is disease states caused by exposure to radiation which may be from an external source or in some cases from intentional application of radiation such as during cancer treatment or x-ray imaging.

It has been discovered that H₂S and SO₂, by their ability to cleave S—S bonds and enter human cells/mitochondria, can treat, inhibit, and/or attenuate disease states by addressing ROS/RSS/RNS abnormalities in cells. It is envisioned that the interaction between H₂S and SO₂ on the one hand and RSS on the other hand leads to the breakdown of RSS to simpler compounds (e.g., those having fewer sulfur atoms). Similarly, it is thought that the interaction between H₂S and SO₂ on the one hand and ROS on the other hand leads to the breakdown of ROS/RSS/RNS into simpler compounds (e.g., those having less diatomic oxygen). The simpler sulfur and oxygen compounds either lead to the formation of normal ROS/RSS/RNS, or deactivation. Deactivated and broken-down ROS/RSS/RNS can be excreted from the body by its normal mechanisms. Overall, these mechanisms work to remediate ROS/RSS/RNS abnormalities and return ROS/RSS/RNS in the body to the normal state. At the same time, H₂S and SO₂ can digest abnormal proteins/DNA/RNA resulting from ROS/RSS/RNS abnormalities allowing them to be excreted from the body. Accordingly, in the case of radiation exposure, treatment with STS serves the triple function of (1) clearing or assisting in the clearance of radioactive metals from the body, (2) addressing ROS/RSS/RNS abnormalities, and (3) addressing the presence of abnormal protein/DNA/RNA resulting from the ROS/RSS/RNS abnormalities, etc. Regarding RNS abnormalities, the H₂S and SO₂ combination can react with O₃ and correct it back to O₂. Furthermore, H₂S converts all abnormal NO's to normal number and formation.

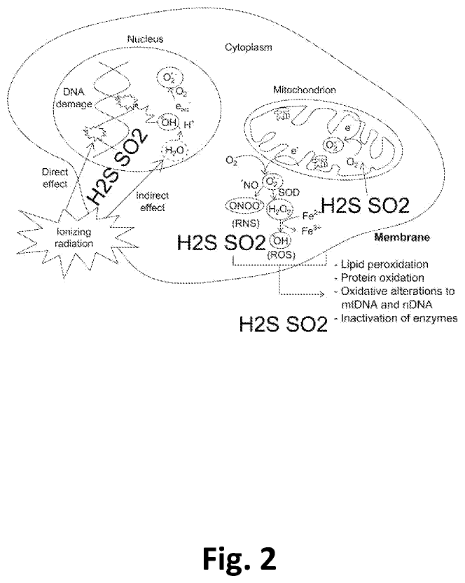

FIG. 2 schematically shows an example human cell 100 including a nucleus 102 and mitochondria 104. DNA 106 is shown inside the nucleus. In this example, ionizing radiation has entered nucleus 102 and created damaged areas 108 of the DNA 106, which either creates mutagenic DNA or non-functional DNA. Moreover, ionizing radiation converts water in the cell to hydroxide ions and oxygen in the mitochondria to hydrogen peroxide and hydroxide ions. Hydroxide ions are ROS that are not normally present in the quantities resulting from the conversion of water and oxygen due to ionizing radiation and can cause problems in cell 100 such as oxidation of proteins and lipids in the cell, oxidative alternations to DNA, and inactivation of enzymes. As shown, both H₂S and SO₂ have entered cell 100, nucleus 102, and mitochondria 104. In this case, if enough H₂S and SO₂ are present in the cell, they can react with the hydroxide ions and hydrogen peroxide and restore them to normal levels. H₂S and SO₂ can also digest damaged DNA preventing it from being replicated and allowing it to be excreted from the body. Therefore, STS treatment can be used to combat the deleterious effect of ionizing radiation on cells.

Another example disease that is characterized by ROS/RSS/RNS abnormalities is retinal diseases, many of which involve oxidative stress conditions. H₂S and/or SO₂ can address such oxidative stress conditions by addressing ROS/RSS/RNS abnormalities as discussed above.

To provide disease treatment, attenuation, or inhibition by regulating and addressing abnormalities associated with ROS, RNS, and/or RSS as discussed above, a composition including STS is administered to a patient. In a particular example, the STS is administered at a dose of between about 1 mg and 2 mg per kg of body weight per day of treatment. The total dose per day of treatment may be administered in a single dose or multiple doses throughout the day.

In some examples, the composition is co-administered with a catalytic component, e.g., one that augments the rate of STS action within the body according to the processes discussed above. In a particular example, the catalytic component is aspirin and/or vitamin C. The catalytic component may be included in the composition including STS or separately.

The delivery of STS to the body may be targeted depending on the source or location of the ROS/RSS/RNS abnormalities if known. For instance, for x-ray imaging, topical STS provides extra protection to the skin which encounters most of the radiation. For other applications, the STS may be ingested and provide systemic treatment.

In some examples, the inhibition, attenuation, or treatment of the disease is accomplished solely by administration of the above-described composition, e.g., without accompanying drugs or other treatments.

Post Nuclear Xray Chemoradiation therapy produces cardiomyopathy heart weaknesses which will be preventable by the STS H2S SO2 anti ION actions.

Although an embodiment of this disclosure has been explained, a worker of ordinary skill in this art would recognize that certain modifications would come within the spirit and scope of this invention.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for restoring, correcting, and/or preventing imbalances in reactive oxygen species, reactive oxygen species, and reactive sulfur species, the imbalances caused by exposure to radiation from radioactive materials, wherein the imbalances result in abnormalities adversely affecting a health of a subject, the abnormalities comprise cell damage, the method comprises:
administering a composition comprising sodium thiosulfate to a patient in need thereof in an effective amount resulting in production of H₂S and SO₂, wherein the H₂S and SO₂ normalize the imbalances.

2. The method of claim 1, wherein the imbalances are excessive increase in production of the reactive oxygen species, reactive sulfur species and the reactive nitrogen species.

3. The method of claim 1, wherein the imbalances are abnormal accumulation of the reactive oxygen species, reactive sulfur species and the reactive nitrogen species in at least nucleus or mitochondria of cells.

4. The method of claim 1, wherein the imbalances are caused by radiation exposure is from a nuclear explosion, nuclear reactor explosion, water contaminated with radio materials, X-ray diagnostics, or food contaminated with radio materials.

5. The method of claim 4, wherein the abnormality is a retinal disease in an adult subject, wherein the H2S and SO2 result in reversal of adult-onset blindness.

6. The method of claim 1, wherein the administering includes administering a dose of between about 1 mg and 2 mg per kg of body weight of the patient per day of treatment.

7. The method of claim 6, wherein the dose is administered as a single dose.

8. The method of claim 6, wherein the dose is administered as multiple doses.

9. The method of claim 1, wherein the composition further comprises a catalytic compound for augmenting a rate of sodium thiosulfate action within the body.

10. The method of claim 9, wherein the sodium thiosulfate and the catalytic compound are administered together.

11. The method of claim 9, wherein the sodium thiosulfate and the catalytic compound are administered separately.

12. The method of claim 9, wherein the catalytic compound is vitamin C.

13. The method of claim 9, wherein the catalytic compound is aspirin.

14. The method of claim 1, wherein the administering includes targeting the delivery of the composition to a source of the abnormality.

15. The method of claim 1, wherein the administering includes administering the composition systemically.

16. The method of claim 1, wherein the composition includes at least one excipient.

17. The method of claim 1, wherein the composition is formulated in a tablet, powder, capsule, solution, cream, paste, or lotion.

18. The method of claim 1, wherein the administering includes delivering the composition intravenously, orally, rectally, sublingually, vaginally, topically, by inhalation, ophthalmic, or intravesical delivery.

19. The method of claim 1, wherein the abnormalities result in leukemia, cancers, mental birth defects, cardiomyopathy.

20. The method of claim 1, wherein the H2S & SO2 are intracellular and intracerebrally.

21. The method of claim 1, wherein the H2S and SO2 normalize the imbalances by repairing repair DNA broken strands or excreting the DNA broken strands.
